(19) United States
(12) Patent Application Publication
(10) Pub. No.: US 2007/0123580 A1
(43) Pub. Date: May 31, 2007

(54) Combination of histone deacetylase inhibitors with chemotherapeutic agents

(76) Inventors: Peter Wisdom Atadja, Acton, MA (US); Stacy William Remiszewski, Washington Township, NJ (US); Nancy Trogani, Boonton, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/557,162

(22) PCT Filed: May 19, 2004
(86) PCT No.: PCT/EP04/05433

§ 371 Date: Jan. 9, 2007

Related U.S. Application Data
(60) Provisional application No. 60/472,161, filed on May 21, 2003.

(51) Int. Cl. A61K 31/405; A61K 31/19

(52) U.S. Cl. 514/419; 514/575

(57) ABSTRACT

The invention relates to a combination which comprises (a) one or more chemotherapeutic agents and (b) a histone deacetylase inhibitor (“HDAI”) for simultaneous, concurrent, separate or sequential use, especially for use in the treatment of proliferative diseases including pre-malignant lesions (e.g. colon polyps) and malignancies, both solid and undifferentiated or other proliferative diseases in a mammal, particularly a human. The invention also relates to pharmaceutical compositions comprising such a combination and to a method of preventing or treating proliferative diseases including pre-malignant lesions (e.g. colon polyps) and malignancies, both solid and undifferentiated or other proliferative diseases, in a mammal, particularly a human, with such a combination. The present invention further also relates to a commercial package or product comprising such a combination.

The invention relates to a combination which comprises (a) one or more chemotherapeutic agents and (b) a histone deacetylase inhibitor (“HDAI”) for simultaneous, concurrent, separate or sequential use, especially for use in the treatment of proliferative diseases including pre-malignant lesions (e.g. colon polyps) and malignancies, both solid and undifferentiated or other proliferative diseases in a mammal, particularly a human. The invention also relates to pharmaceutical compositions comprising such a combination and to a method of preventing or treating proliferative diseases including pre-malignant lesions (e.g. colon polyps) and malignancies, both solid and undifferentiated or other proliferative diseases, in a mammal, particularly a human, with such a combination. The present invention further also relates to a commercial package or product comprising such a combination.

### BACKGROUND

Reversible acetylation of histones is a major regulator of gene expression that acts by altering accessibility of transcription factors to DNA. In normal cells, histone deacetylase (HDA) and histone acetyltrasferase together control the level of acetylation of histones to maintain a balance. Inhibition of HDA results in the accumulation of hyperacetylated histones, which results in a variety of cellular responses. Inhibitors of HDA (HDAI) have been studied for their therapeutic effects on cancer cells. Recent developments in the field of HDAI research have provided active compounds, both highly efficacious and stable, that are suitable for treating tumors.

Accruing evidence suggests that HDAI are even more efficacious when used in combination with other chemotherapeutic agents. There are both synergistic and additive advantages, both for efficacy and safety. Therapeutic effects of combinations of chemotherapeutic agents with HDAI can result in lower safe dosages ranges of each component in the combination.

The Diseases to be Treated

The combinations of the present invention are useful for treating proliferative diseases. A proliferative disease is mainly a tumor disease (or cancer) (and/or any metastases). The inventive combinations are particularly useful for treating a tumor which is a breast cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example, a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemotherapeutics; or (iii) a tumor that is refractory to treatment with other chemotherapeutics due to multidrug resistance.

In a broader sense of the invention, a proliferative disease may furthermore be a hyperproliferative condition, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Other malignancies which may be treated according to this invention includes a malignancy that is susceptible to treatment with an HDAI compound, for example, breast cancer, lung cancer, ovarian cancer, lymphoma, head and neck cancer and cancer of the colon, esophagus, stomach, bladder, prostrate, uterus and cervix.

Where a tumor, a tumor disease, a carcinoma or a cancer are mentioned, also metastasis in the original organ or tissue and/or in any other location are implied alternatively or in addition, whatever the location of the tumor and/or metastasis.

The Chemotherapeutic Agents

The term “chemotherapeutic agent(s)” is a broad one, as there are many cancer chemotherapeutic agents, having different mechanisms of action. Combinations of these with HDAI agents can result in improvements in cancer therapy. Generally, chemotherapeutic agents are classified according to the mechanism of action. Many of the available agents are antimetabolites of development pathways of various tumors, or react with the DNA of the tumor cells. There are also agents which inhibit enzymes such as topoisomerase I and topoisomerase II, or which are antimiotic agents.

By the term “chemotherapeutic agent” is meant especially any chemotherapeutic agent other than a histone deacetylase inhibitor (“HDAI”) or a derivative thereof. It includes but is not limited to,
i. an aromatase inhibitor,
ii. an antiestrogen, an anti-androgen (especially in the case of prostate cancer) or a gonadorelin agonist,
iii. a topoisomerase I inhibitor or a topoisomerase II inhibitor,
iv. a microtubule active agent, an alkylating agent, an antineoplastic antimetabolite or a platin compound,
v. a compound targeting/decreasing a protein or lipid kinase activity or a protein or lipid phosphatase activity, a further anti-angiogenic compound or a compound which induces cell differentiation processes,
vi. a bradykinin 1 receptor or an angiotensin II antagonist,
vii. a cyclooxygenase inhibitor, a bisphosphonate, a rapamycin derivative such as everolimus, a heparanase inhibitor (prevents heparan sulphate degradation), e.g. PI-88, a biological response modifier, preferably a lymphokine or interferons, e.g. interferon γ, an ubiquitination inhibitor, or an inhibitor which blocks anti-apoptotic pathways,
viii. an inhibitor of Ras oncogenic isoforms, e.g. H-Ras, K-Ras or N-Ras, or a farnesyl transferase inhibitor, e.g. L-744,832 or DK8G557,
ix. a telomerase inhibitor, e.g. telomestatin,
x. a protease inhibitor, a matrix metalloproteinase inhibitor, a methionine aminopeptidase inhibitor, e.g. bengamide or a derivative thereof, or a proteosome inhibitor, e.g. PS-341.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™ Aminoglutethimide can be administered, e.g., in the form as it is marketed, e.g. under the trademark ORIMETEN™. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., In the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX™), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, e.g. as disclosed in U.S. Pat. No. 5,843,901.

The term “topolsomerase I inhibitor” as used herein includes, but is not limited to topotecan, irinotecan, 9-nitrocamptothecin, 7-(t-butoxy)imino methyl camptothecin (gimatecan) and the macromolecular camptothecin conjugate PNU-166148 (compound A1 in WO99/17804). Irinotecan can be administered, e.g. in the form as it is marketed, e.g. under the trademark CAMPTOSAR™. Topotecan can be administered, e.g., in the form as it is marketed, e.g. under the trademark HYCAMTIN™.

The term “topoisomerase II inhibitor” as used herein includes, but is not limited to the anthracyclines such as doxorubicin (including liposomal formulation, e.g. CAELYX™), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS™. Teniposide can be administered, e.g. in the form as it is marketed, e.g. under the trademark VM 26BRISTOL™ Doxorubicin can be administered, e.g. in the form as it is marketed, e.g. under the trademark ADRIBLASTIN™. Epirubicin can be administered, e.g. in the form as it is marketed, e.g. under the trademark FARMORUBICIN™. Idarubicin can be administered, e.g. in the form as it is marketed, e.g. under the trademark ZAVEDOS™. Mitoxantrone can be administered, e.g. in the form as it is marketed, e.g. under the trademark NOVANTRON™.

The term “microtubule active agent” relates to microtubule stabilizing and microtubule destabilizing agents including, but not limited to taxanes, e.g. paclitaxel and docetaxel, vinca alkaloids, e.g., vinblastine, especially vinblastine sulfate, vincristine especially vincristine sulfate, and vinorelbine, discodermolides and epothilones and derivatives thereof, e.g. epothilone B or a derivative thereof. Paclitaxel may be administered e.g. in the form as it is marketed, e.g. TAXOL™. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agent” as used herein includes, but is not limited to cyclophosphamide, ifosfamide, melphalan or nitrosourea (BCNU or Gliadel™). Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “antineoplastic antimetabolite” includes, but is not limited to 5-fluorouracil, capecitabine, gemcitabine, DNA demethylating agents, such as 5-azacytidine and decitabine, methotrexate and edatrexate. Capecitabine can be administered, e.g., in the form as it is marketed, e.g. under the trademark XELODA™. Gemcitabine can be administered, e.g., in the form as it is marketed, e.g. under the trademark GEMZAR™.

The term “platin compound” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds targeting/decreasing a protein or lipid kinase activity or further anti-angiogenic compounds” as used herein includes, but is not limited to protein tyrosine kinase and/or serine and/or threonine kinase inhibitors or lipid kinase inhibitors, e.g. compounds targeting, decreasing or inhibiting the activity of the epidermal growth factor family of receptor tyrosine kinases (EGFR, ErbB2, ErbB3, ErbB4 as homo- or heterodimers), the vascular endothelial growth factor family of receptor tyrosine kinases (VEGFR), the platelet-derived growth factor-receptors (PDGFR), the fibroblast growth factor-receptors (FGFR), the insulin-like growth factor receptor 1 (IGF-1R), the Trk receptor tyrosine kinase family, the Axl receptor tyrosine kinase family, the Ret receptor tyrosine kinase, the Kit/SCFR receptor tyrosine kinase, members of the c-Abl family and their gene-fusion products (e.g. BCR-Abl), members of the protein kinase C (PKC) and Raf family of serine/threonine kinases, members of the MEK, SRC, JAK, FAK, PDK or PI(3) kinase family, or of the PI(3)-kinase-related kinase family, and/or members of the cyclin-dependent kinase family (CDK) and anti-angiogenic compounds having another mechanism for their activity, e.g. unrelated to protein or lipid kinase inhibition.

Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; Endostatin™, described by M. S. O'Reilly et al, Cell 88, 1997, 277-285; anthranilic acid amides; ZD4190; ZD6474; SU5416; SU6668; or anti-VEGF antibodies or anti-VEGF receptor antibodies, e.g. RhuMab.

By antibody is meant intact monoclonal antibodies, polyclonal antibodies, multispecific antibodies formed from at least 2 intact antibodies, and antibodies fragments so long as they exhibit the desired biological activity.

Compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herpetin®), cetuximab, Iressa, OSI-774, CI-1033, EKB-569, GW-2016, E1.1, E2.4, E2.5, E6.2, E6.4, E2.11, E6.3 or E7.6.3.

Compounds which target, decrease or inhibit the activity of PDGFR are especially compounds which inhibit the PDGF receptor, e.g. a N-phenyl-2-pyrimidine-amine derivative, e.g. imatinib.

Compounds which target decrease or inhibit the activity of c-Abl family members and their gene fusion products, e.g. a N-phenyl-2-pyrimidine-amine derivative, e.g. imatinib; PD180970; AG957; or NSC 680410.

Compounds which target, decrease or inhibit the activity of protein kinase C, Raf, MEK, SRC, JAK, FAK and PDK family members, or PI(3) kinase or PI(3) kinase-related family members, and/or members of the cyclin-dependent kinase family (CDK) are especially those staurosporine derivatives disclosed in EP 0 296 110, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; or LY333531/LY379196.

Further anti-angiogenic compounds are e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof. Compounds which induce cell differentiation processes are e.g. retinoic acid, α-, γ- or δ-tocopherol or α-, γ- or δ-tocotrienol.

The term cyclooxygenase inhibitor as used herein includes, but is not limited to, e.g. celecoxib (Celebrex®), rofecoxib (Vioxx®), etoricoxib, valdecoxib or a 5-alkyl-2-arylaminophenylacetic acid, e.g. 5-methyl-2-(2′-chloro-6′-fluoroanilino)phenyl acetic acid.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g. in the form as it is marketed, e.g. under the trademark ZOMETA™

The term “matrix metalloproteinase inhibitor” as used herein includes, but is not limited to collagen peptidomimetic and nonpeptidomimetic inhibitors, tetracycline derivatives, e.g. hydroxamate peptdomimetic inhibitor batimastat and its orally bioavailable analogue marimastat, prinomastat, BMS-279251, BAY 12-9566, TAA211 or AAJ996.

It has been found that the following classes of chemotherapeutic agents are especially useful in the practice of this invention: antimetabolites, DNA topoisomerase I inhibitors, DNA topoisomerase II inhibitors, microtubule active agents, and the like. Within the class of antimetabolites are included agents that are inhibitors of thymidine production; inhibitors of vascular endothethial growth factor; DNA demethylating agents; or protein-tyrosine kinase inhibitors.

Representative examples of these classes of chemotherapeutic agents which are useful in the practice of this invention are illustrated as follows:

DNA topoisomerase I inhibitors: camptothecin or derivatives thereof such as gimatecan.

DNA topoisomerase I inhibitors: Adriamycin

Microtubule active agents: Discodermolides and epothilones such as epothilone B and epothilone D.

Antimetabolites, including:
Thymidine production inhibitors, such as 5-Fluorouracil;
DNA demethylating agents, such as 5-Azacybidine and decitabine;
Vascular endothethial growth factor inhibitors, such as 1-[4-chloroanilino]-4-[pyridylmethyl]-phthalazine succinate (PTK 787);
Protein-tyrosine kinase inhibitors, such as imatinib mesylate (Gleevec).

Generally speaking the chemotherapeutic agents useful in the combinations of this invention, viz., any of the chemotherapeutic agents mentioned above, and especially those which are DNA topoisomerase II inhibitors, microtubule active agents, thymidine production inhibitors, DNA topoisomerase I inhibitors, and DNA demethylating agents, are used together with the HDAI compounds in the dosages and with the therapeutic regimes as employed in the usual course of therapy of each drug alone.

The HDAI Compounds

HDAI compounds of particular interest for use in the inventive combinations are hydroxamate compounds described by the formula (I) 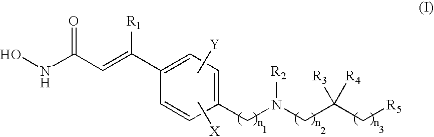
wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (preferably C₁-C₆ alkyl, e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₆, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
   or a pharmaceutically acceptable salt thereof.

As appropriate, unsubstituted means that there is no substituent or that the only substituents are hydrogen.

Halo substituents are selected from fluoro, chloro, bromo and iodo, preferably fluoro or chloro.

Alkyl substituents include straight and branched C₁-C₆alkyl, unless otherwise noted. Examples of suitable straight and branched C₁-C₆alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e. there are one or more double or triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing from one to three heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and OR₁₅, for example alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula —(CH₂)ₙ₅-cycloalkyl wherein n5 is a number from 1-6. Suitable cycloalkylalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and OR₁₅, such as alkoxy. Preferred substituents include-including C₁-C₆ alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, cycloalkylalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR₁₅, such as alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, for example by R₁₃; especially useful N substituents include H, C₁-C₄ alkyl, acyl, aminoacyl, and sulfonyl.

Arylalkyl substituents include groups of the formula —(CH₂)ₙ₅-aryl, —(CH₂)ₙ₅₋₁—(CH-aryl)-(CH₂)ₙ₅-aryl or —( CH₂)ₙ₅₋₁CH(aryl)(aryl) wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl wherein heteroaryl and n5 are defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅ wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±-3-amin-4-hexenoyl.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, octahydroindene, perhydrobenzocycloheptene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatoms, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, for example by R₁₃; especially useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatoms, for example, 1, 2, 3, or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatoms chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydropyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W, and —N(R₁₂)C(O)—NHOH and R₁₂ and W are defined above.

The R₂ substituent HON—C(O)—CH═C(R₁)-aryl-alkyl- is a group of the formula

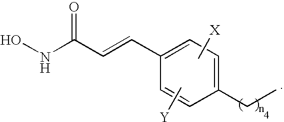

Preferences for each of the substituents include the following:
R₁ is H, halo, or a straight chain C₁-C₄ alkyl;
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl, and —(CH₂)ₙR₇;
R₃ and R₄ are the same or different and independently selected from H, and C₁-C₆ alkyl, or R₃ and R₄ together with the carbon to which they are bound represent C═O, C═S, or C═NR₆;
R₅ is selected from H, C₁-C₆ alkyl, C₄-C₉ cycloalkyl, C₄-C₉ heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄ alkyl, CF₃, NO₂, C(O)R₁, OR₉, SR₉, CN, and NR₁₀R₁₁;
R₆ is selected from H, C₁-C₆ alkyl, C₄-C₉ cycloalkyl, C₄₋₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁, are the same or different and independently selected from H, C₁-C₄ alkyl, and —C(O)-alkyl;
R₁₂ is selected from H, C₁-C₆ alkyl, C₄-C₁₀ cycloalkyl, C₄-C₉ heterocycloalkyl, aryl, heteroaryl, arylalkyl, and heteroarylalkyl;
R₁₃ and R₁₄ are the same or different and independently selected from H, C₁-C₆ alkyl, C₄-C₉ cycloalkyl, C₄-C₁₀ heterocycloalkyl, aryl, heteroaryl, arylalkyl, heteroarylalkyl and amino acyl;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₇ is selected from C₁-C₈ alkyl, C₄-C₉ cycloalkyl, C₄-C₉ heterocycloalkyl, aryl, heteroaryl, arylalkyl, heteroarylalkyl and NR₁₃R₁₄;
m is an integer selected from 0 to 6; and
Z is selected from O, NR₁₃, S, S(O),
   or a pharmaceutically acceptable salt thereof.

Useful compounds of the formula (I) include those wherein each of R₁, X, Y, R₃, and R₄ is H, including those wherein one of n₂ and n₃ is zero and the other is 1, especially those wherein R₂ is H or —CH₂—CH₂—OH.

One suitable genus of hydroxamate compounds are those of formula (Ia) 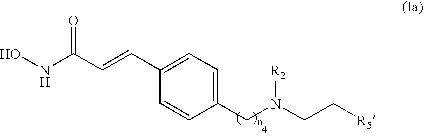
wherein
n₄ is 0-3,
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is heteroaryl, heteroarylalkyl (e.g., pyridylmethyl), aromatic polycycles, non-aromatic polycycles, mixed aryl and non-aryl polycycles, polyheteroaryl, or mixed aryl and non-aryl polyheterocycles,
   or a pharmaceutically acceptable salt thereof.

Another suitable genus of hydroxamate compounds are those of formula (Ia)

wherein

n₄ is 0-3,
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is aryl, arylalkyl, aromatic polycycles, non-aromatic polycycles, and mixed aryl and non-aryl polycycles; especially aryl, such as p-fluorophenyl, p-chlorophenyl, p-O—C₁-C₄-alkylphenyl, such as p-methoxyphenyl, and p-C₁-C₄-alkylphenyl; and arylalkyl, such as benzyl, ortho, meta or para-fluorobenzyl, ortho, meta or para-chlorobenzyl, ortho, meta or para-mono, di or tri-O—C₁-C₄-alkylbenzyl, such as ortho, meta or para-methoxybenzyl, m,p-diethoxybenzyl, o,m,p-triimethoxybenzyl, and ortho, meta or para-mono, di or tri C₁-C₄-alkylphenyl, such as p-methyl, m,m-diethylphenyl,
   or a pharmaceutically acceptable salt thereof.

Another interesting genus is the compounds of formula (Ib) 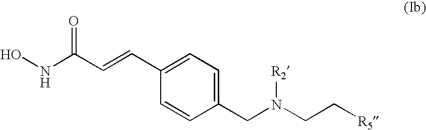
wherein
R2′ is selected from H, C₁-C₆ alkyl, C₄-C₆ cycloalkyl, cycloalkylalkyl (e.g., cyclopropylmethyl), (CH₂)₂₋₄OR₂₁ where R₂₁ is H, methyl, ethyl, propyl, and i-propyl, and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl,
or a pharmaceutically acceptable salt thereof.

Another interesting genus of hydroxamate compounds are the compounds of formula (Ic) 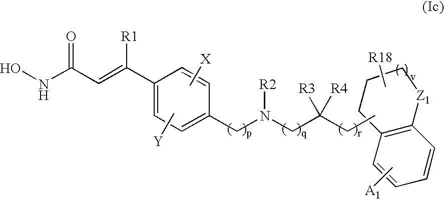
wherein
the ring containing Z. is aromatic or non-aromatic, which non-aromatc rings are saturated or unsaturated,
Z₁ is O, S or N—R₂₀,
R18 is H, halo, C₁-C₆alkyl (methyl, ethyl, t-butyl), C₃-C₇cycloalkyl, aryl, for example unsubstituted phenyl or phenyl substituted by 4-OCH₃ or 4-CF₃, or heteroaryl, such as 2-furanyl, 2-thiophenyl or 2-, 3- or 4-pyridyl;
R₂₀ is H, C₁-C₆alkyl, C₃-C₉cycloalkyl-C₁-C₆alkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl);
A₁ is 1, 2 or 3 substituents which are independently H, C₁-C₆alkyl, —OR₁₉, halo, alkylamino, aminoalkyl, halo, or heteroarylalkyl (e.g., pyridylmethyl);
R₁₉ is selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl) and —(CH₂CH═CH(CH₃)(CH₂))₁₋₃H;
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2,
p is 0-3, and
q is 1-5 and r is 0 or
q is 0 and r is 1-5,
   or a pharmaceutically acceptable salt thereof. The other variable substituents are as defined above.

Especially useful compounds of formula (Ic) are those wherein R₂ is H, or —(CH₂)ₙCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id) 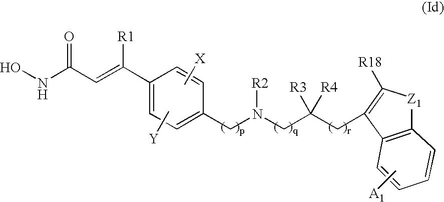
wherein
Z₁ is O, S or N—R₂₀,
R18 is H, halo, C₁-C₆alkyl (methyl, ethyl, t-butyl), C₃-C₇cycloalkyl, aryl, for example, unsubstituted phenyl or phenyl substituted by 4-OCH₃ or 4-CF₃, or heteroaryl,
R₂₀ is H, C₁-C₆alkyl, C₃-C₉cycloalkyl-C₁-C₆alkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl),
A₁ is 1, 2 or 3 substituents which are independently H, C₁-C₆alkyl, —OR₁₉, or halo,
R₁₉ is selected from H, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
p is 0-3, and
q is 1-5 and r is 0 or
q is 0 and r is 1-5,
or a pharmaceutically acceptable salt thereof. The other variable substituents are as defined above.

Especially useful compounds of formula (Id) are those wherein R₂ is H. or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Further very interesting HDAI compounds for use according to the present invention are compounds of the formula (Ie) 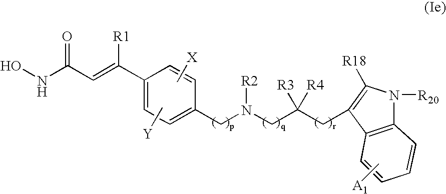
or a pharmaceutically acceptable salt thereof. The variable substituents are as defined above.

Especially useful compounds of formula (Ie) are those wherein R18 is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl (e.g., pyridyl) ring.

Another group of useful compounds of formula (Ie) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R3 and R4 are preferably H.

Another group of useful compounds of formula (Ie) are those wherein R18 is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H, or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie) wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof, are important compounds of formula (Ie).

Another suitable genus of hydroxamate compounds are those of formula (If) 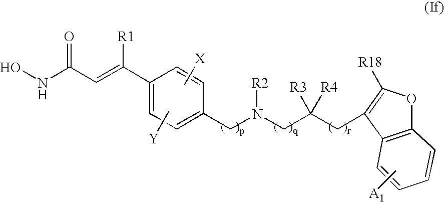
or a pharmaceutically acceptable salt thereof. The variable substituents are as defined above.

Useful compounds of formula (If) are include those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide, or a pharmaceutically acceptable salt thereof, is an important compound of formula (If).

Very preferred histone deacetylase inhibitors for use according to the present invention are the compounds disclosed in the examples of WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof or a pharmaceutically acceptable prodrug thereof, most preferably N-hydroxy-3-[4-[[(2-hydroxyethyl)[2-(1H-indol-3-yl)ethyl]-amino]methyl]phenyl]-2E-2-propenamide and N-hydroxy-3-[4-[[[2-(2-methyl-1H-indol-3-yl)ethyl]-amino]methyl]phenyl]-2E-2-propenamide, or a pharmaceutically acceptable salt thereof or a pharmaceutically acceptable prodrug thereof.

The histone deacetylase inhibitors mentioned hereinbefore can be prepared according to the processes described in WO 02/22577.

In addition to the prevention and treatment of proliferative diseases including pre-malignant lesions as well as both solid and undifferentiated malignancies, such as pre-malignant colon lesions (e.g. polyps) and colon cancer, the inventive combination therapy has utility for the treatment of “other malignancies”, which is hereby defined as a malignancy that is susceptible to treatment with an HDAI compound, for example, breast cancer, lung cancer, ovarian cancer, lymphoma, head and neck cancer and cancer of the esophagus, stomach, bladder, prostrate, uterus and cervix.

The Combinations

Thus, in a first aspect, the present invention relates to a combination which comprises (a) a chemotherapeutic agent, especially selected from those mentioned herein, most especially from those mentioned as being preferred, and (b) a histone deacetylase inhibitor, especially selected from those mentioned herein, most especially from those mentioned as being preferred, in which (a) and (b) are present in each case in free form or in the form of a pharmaceutically acceptable salt or a pharmaceutically acceptable prodrug thereof, for simultaneous, concurrent, separate or sequential use.

In another embodiment the invention relates a combination which comprises (a) a chemotherapeutic agent, especially selected from those mentioned herein, most especially from those mentioned as being preferred, and (b) a histone deacetylase inhibitor, especially selected from those mentioned herein, most especially from those mentioned as being preferred, in which (a) and (b) are present in each case in free form or in the form of a pharmaceutically acceptable salt or a pharmaceutically acceptable prodrug thereof, for use in the treatment of the human or animal body, especially for the treatment of a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

The present invention also relates to the use of a histone deacetylase inhibitor, especially selected from those mentioned herein, most especially from those mentioned as being preferred, or a pharmaceutically acceptable salt thereof or a pharmaceutically acceptable prodrug thereof, for the preparation of a medicament, for use in combination with a chemotherapeutic agent, especially selected from those mentioned herein, most especially from those mentioned as being preferred, or a pharmaceutically acceptable salt thereof or a pharmaceutically acceptable prodrug thereof, for the treatment of a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

The invention further also relates to the use of a chemotherapeutic agent, especially selected from those mentioned herein, most especially from those mentioned as being preferred, or a pharmaceutically acceptable salt thereof or a pharmaceutically acceptable prodrug thereof, for the preparation of a medicament, for use in combination with a histone deacetylase inhibitor, especially selected from those mentioned herein, most especially from those mentioned as being preferred, or a pharmaceutically acceptable salt thereof or a pharmaceutically acceptable prodrug thereof, for the treatment of a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

In another embodiment the present invention relates to a pharmaceutical composition which comprises (a) a chemotherapeutic agent, especially selected from those mentioned herein, most especially from those mentioned as being preferred, or a pharmaceutically acceptable salt thereof or a pharmaceutically acceptable prodrug thereof, and (b) a histone deacetylase inhibitor, especially selected from those mentioned herein, most especially from those mentioned as being preferred, or a pharmaceutically acceptable salt thereof or a pharmaceutically acceptable prodrug thereof, together with at least one pharmaceutically acceptable carrier.

In a further embodiment the invention relates to the use of a combination which comprises (a) a chemotherapeutic agent, especially selected from those mentioned herein, most especially from those mentioned as being preferred, and (b) a histone deacetylase inhibitor, especially selected from those mentioned herein, most especially from those mentioned as being preferred, in which (a) and (b) are present in each case in free form or in the form of a pharmaceutically acceptable salt or a pharmaceutically acceptable prodrug thereof, for the preparation of a pharmaceutical composition for the treatment of a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

In another embodiment the present invention relates to a commercial package or product comprising a chemotherapeutic agent, especially selected from those mentioned herein, most especially from those mentioned as being preferred, or a pharmaceutically acceptable salt thereof or a pharmaceutically acceptable prodrug thereof, together with instructions for use in combination with a histone deacetylase inhibitor, especially selected from those mentioned herein, most especially from those mentioned as being preferred, or a

pharmaceutically acceptable salt thereof or a pharmaceutically acceptable prodrug thereof, for the treatment of a disease in a mammal, or a commercial package or product comprising a histone deacetylase inhibitor, especially selected from those mentioned herein, most especially from those mentioned as being preferred, or a pharmaceutically acceptable salt thereof or a pharmaceutically acceptable prodrug thereof, together with instructions for use in combination with a chemotherapeutic agent, especially selected from those mentioned herein, most especially from those mentioned as being preferred, or a pharmaceutically acceptable salt thereof or a pharmaceutically acceptable prodrug thereof, for the treatment of a disease in a mammal; wherein in both cases the disease to be treated is especially a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

The invention also relates to a commercial package or product comprising a combination which comprises (a) a chemotherapeutic agent, especially selected from those mentioned herein, most especially from those mentioned as being preferred, and (b) a histone deacetylase inhibitor, especially selected from those mentioned herein, most especially from those mentioned as being preferred, in which (a) and (b) are present in each case in free form or in the form of a pharmaceutically acceptable salt or a pharmaceutically acceptable prodrug thereof, together with instructions for simultaneous, concurrent, separate or sequential use thereof in the treatment of a disease in a mammal, especially a proliferative disease, preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single patient based on the severity of any side-effects that the patient experiences.

The present invention therefore also relates to a combined preparation which comprises (a) one or more unit dosage forms of a chemotherapeutic agent, especially selected from those mentioned herein, most especially from those mentioned as being preferred, or a pharmaceutically acceptable salt thereof or a pharmaceutically acceptable prodrug thereof, and (b) one or more unit dosage forms of a histone deacetylase inhibitor, especially selected from those mentioned herein, most especially from those mentioned as being preferred, or a pharmaceutically acceptable salt thereof or a pharmaceutically acceptable prodrug thereof.

The invention further also relates to a method for the prevention or treatment of proliferative diseases including pre-malignant lesions as well as both solid and undifferentiated malignancies in a mammal, which comprises treating the mammal with pharmaceutically effective amounts of a combination which comprises (a) a chemotherapeutic agent, especially selected from those mentioned herein, most especially from those mentioned as being preferred, and (b) a histone deacetylase inhibitor, especially selected from those mentioned herein, most especially from those mentioned as being preferred, in which (a) and (b) are present in each case in free form or in the form of a pharmaceutically acceptable salt or a pharmaceutically acceptable prodrug thereof, wherein the proliferative disease is preferably a proliferative disease mentioned herein, most preferably a disease selected from the group consisting of breast cancer, lung cancer, ovarian cancer, lymphoma, head and neck cancer and cancer of the esophagus, stomach, bladder, prostrate, uterus and cervix.

In a specific embodiment, the disease to be treated with a combination of the present invention is colon cancer. In another embodiment, the disease to be treated is pre-malignant colon lesions. In another embodiment, the diseases to be treated are proliferative diseases including pre-malignant lesions as well as both solid and undifferentiated malignancies as described above.

According to the present invention, a patient is treated simultaneously, concurrently, separately or sequentially with pharmaceutically effective amounts of a combination of a chemotherapeutic agent and an HDAI in order to prevent or treat proliferative diseases including pre-malignant lesions as well as both solid and undifferentiated malignancies including pre-malignant colon lesions, such as polyps, or colon cancer, or another malignancy, each according to a dosage regimen that is appropriate for the individual agent. For example, the chemotherapeutic agent may be administered once or more daily and the HDAI may be administered once daily, on alternate days or on some other schedule—as is appropriate for the HDAI agent when used without the chemotherapeutic agent. One of skill in the art has the ability to determine appropriate pharmaceutically effective amounts of the combination components.

In the instance where the chemotherapeutic agent is selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, Discodermolides and epothilones, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; pharmaceutically acceptable salts thereof; and pharmaceutically acceptable prodrugs, e.g. esters, thereof; and the patient to be treated is a human, an appropriate dose of e.g., Adriamycin is in the range from 100 to 1500 mg daily, for example, 200-1000 mg/day, such as 200, 400, 500, 600, 800, 900 or 1000 mg/day, administered in one or two doses daily. 5-Fluorouracil is administered at a appropriate dose in the range from 100 to 1500 mg daily, for example, 200-1000 mg/day, such as 200, 400, 500, 600, 800, 900 or 1000 mg/day, administered in one or two doses daily. Camptothecin or derivatives thereof are administered at an appropriate dose in the range from 100 to 1500 mg daily, for example, 200-1000 mg/day, such as 200, 400, 500, 600, 800, 900 or 1000 mg/day, administered in one or two doses daily. 5-Azacytidine is administered at a appropriate dose in the range from 100 to 1500 mg daily, for example, 200-1000 mg/day, such as 200, 400, 500, 600, 800, 900 or 1000 mg/day, administered in one or two doses daily. Preferably, the compounds or the pharmaceutically acceptable salts thereof, are administered as an oral pharmaceutical formulation in the form of a tablet, capsule or syrup; or as parenteral injections if appropriate.

The combination partner (a) or (b), or a pharmaceutically acceptable salt thereof or a pharmaceutically acceptable prodrug thereof, may also be used in form of a hydrate or other solvate.

The following Example illustrates the invention described above; it is not, however, intended to limit the scope of the invention in any way. The beneficial effects of the combination of the invention can also be determined by other test models known as such to the person skilled in the pertinent art.

### EXAMPLES

The chosen chemotherapeutic agent selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, Discodermolides and epothilones, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; pharmaceutically acceptable salts thereof; and pharmaceutically acceptable prodrugs, e.g. esters, thereof; (“chemotherapeutic agent”) and N-hydroxy-3-[4-[[(2-hydroxyethyl)[2-(1H-indol-3-yl)ethyl]-amino]methyl]phenyl]-2E-2-propenamide (alternatively named (E)-N-Hydroxy-3-[4-({(2-hydroxy-ethyl)-[2-(1H-indol-3-yl)-ethyl]-amino}-methyl)phenyl]-acrylamide) (“HDAI”) are tested as single agents and together as combination therapy in a mouse model of adenomatous polyposis for the prevention and treatment of intestinal polyps. HDAI is administered intravenously to the mice at 10 mg/kg as a solution in D5W containing a pharmaceutically acceptably acid, e.g., lactic acid, adjusted to a pH appropriate for administration to an animal or human, e.g. pH from 3.5 to 5.5, q.d., 3 times per week for three weeks. The chemotherapeutic agent is administered as a dietary or injectable admixture at the appropriate concentration.

The effects of HDA inhibitors individually or in combination with standard chemotherapeutic agents are tested in vitro to gain insights into possible adverse effects and the potential clinical use of one HDAI compound, N-hydroxy-3-[4-[[(2-hydroxyethyl)[2-(1H-indol-3-yl)ethyl]-amino]methyl]phenyl]-2E-2-propenamide, in combination chemotherapy.

MTS (which is 3-(4,5 Dimethylthiazol-2-yl)-5-(3-carboxyphenyl)-2-(4-sulfophenyl)-2H-tetrazolium, inner salt) assays are used to assess the antiproliferative effects of the drug combinations. The HCT116 and MDA-MB-435P tumor cell lines, previously characterized as sensitive to HDA inhibitors, are chosen for these experiments along with clinically relevant chemotherapeutic drugs. A commercial program, “CalcuSyn”, is employed to determine whether the combined effects are synergistic, antagonistic or additive.

When cells are treated with adriamycin 24 hr prior to N-hydroxy-3-[4-[[(2-hydroxyethyl)[2-(1H-indol-3-yl)ethyl]-amino]methyl]phenyl]-2E-2-propenamide, synergistic effects are obtained.

Combination of N-hydroxy-3-[4-[[(2-hydroxyethyl)[2-(1H-indol-3-yl)ethyl]-amino]methyl]phenyl]-2E-2-propenamide with 5-FU, camptothecin or 5-azacytidine produced mainly additive effects.

N-hydroxy-3-[4-[[(2-hydroxyethyl)[2-(1H-indol-3-yl)ethyl]-amino]methyl]phenyl]-2E-2-propenamide is a structurally novel, HDAI that has been demonstrated to have potent anti-tumor activity in vitro and in vivo. This compound increases histone acetylation, transcriptionally activates the p21 promoter and inhibits cell growth at sub-micromolar concentrations. Whereas N-hydroxy-3-[4[[(2-hydroxyethyl)[2-(1H-indol-3-yl)ethyl]-amino]methyl]phenyl]-2E-2-propenamide produces apoptosis in HCT116 cells at sub micromolar levels after 48 to 72 hours of exposure, normal dermal fibroblasts predominantly undergo cell-cycle arrest and much higher concentrations and longer times of exposure are required to reduce their viability.

N-hydroxy-3-[4-[[(2-hydroxyethyl)[2-(1H-indol-3-yl)ethyl]-amino]methyl]phenyl]-2E-2-propenamide reproducibly inhibits tumor growth in the HCT116 colon, A549 lung, and MDA-M B-435P breast carcinoma xenograft models with single daily intravenous doses ranging from 10 to 100 mg/kg. Increased levels of histone acetylation are observed in HCT116 cells treated in culture continuously for 3 to 24 hr in the presence of N-hydroxy-3-[4-[[(2-hydroxyethyl)[2-(1H-indol-3-yl)ethyl]-amino]methyl]phenyl]-2E-2-propenamide, indicating intracellular HDA inhibition. The compound also increases acetylated histone H3 and H4 levels in vivo, in HCT116 human tumor xenografts from athymic mice. In this example, the effects of combining N-hydroxy-3-[4-[[(2-hydroxyethyl)[2-(1H-indol-3-yl)ethyl]-amino]methyl]phenyl]-2E-2-propenamide with standard therapeutics or with other experimental drugs on monolayer proliferation of tumor cell lines are investigated.

Two cell lines, the HCT116 human colon carcinoma cells and MDA-MB-435P, a hormone independent breast carcinoma cell line, are chosen based on their sensitivity to HDAI. Chemotherapeutic agents are paired with the cell lines based on their clinical relevance. The compounds tested in combination with N-hydroxy-3-[4-[[(2-hydroxyethyl)[2-(1H-indol-3-yl)ethyl]-amino]methyl]phenyl]-2E-2-propenamide include adriamycin which is clinically used in the treatment of breast cancer, 5-fluoruracil (5-FU), and camptothecin, used clinically for colon cancer. Due to the reported cross-talk between histone deacetylases and DNA methyl transferases, 5-azacytidine, a DNA methyltransferase inhibitor, is also tested.

HCT116 cells are obtained from ATCC and cultured as previously described, Alley M C, Scudiero D A, Monks A, Hursey, M L, Czerwinski, M J, Fine D L, Abbot B J, Mayo J G, Shoemaker R H, Boyd M R, “Feasibility of drug screening with panels of human tumor cell lines using a microculture tetrazolium assay”, Cancer Res. 1988; 48:589-601.

Human breast carcinoma cells, MDA-MB-435P are cultured as previously described in the reference above. Cell proliferation is measured using an adaptation of published procedures essentially as described, in Zhang R D, Fidler I J, Price J E, “Relative malignant potential of human breast carcinoma cell lines established from pleural effusions and a brain metastasis”, Invasion-Metastasis 1991; 11 :204-15.

Experiments are done using five-point or six-point drug titrations in 96-well tissue culture plates, with the top row left empty. HCT116 and MDA-MB-435P cells are suspended in complete media at a density of 3.6×10³ and 2.1×10⁴ cell/ml, respectively, and 190 μl are added per well. Complete medium (200 μl) is added to the top row. 24 hours later, after the cells have attached to the bottom of the plate, 10 μl of MTS solution are added to one of the plates to determine the activity at the time of compound addition (T₀). The plate is incubated at 37° C. for 4 hours and the OD is measured on a Molecular Devices Thermomax at 490 nm using the Softmax program. The T₀ plate serves as a reference for initial activity at the beginning of the experiment. Compound addition begins 24 hours after seeding, the same time as the T₀ determination. Serial dilutions (as suggested by the CalcuSyn program) at 4-fold, 2-fold, 1-fold, 0.5-fold, 0.25-fold and 0.125-fold of previously determined IC₅₀ values of each compound are made in a 96-deep well plate with the highest concentrations on the edge of the plate. One cell line is tested with four compounds or combinations per plate. 10 microliters of each of the six dilutions are added in triplicate and complete medium is added to the bottom row. The compounds are added to the plates singly or in combination with N-hydroxy-3-[4-[[(2-hydroxyethyl)[2-(1H-indol-3-yl)ethyl]-amino]methyl]phenyl]-2E-2-propenamide. In pretreated combinations, the drugs are added simultaneously, 24 hr prior, or 24 hr after N-hydroxy-3-[4-[[(2-hydroxyethyl)[2-(1H-indol-3-yl)ethyl]-amino]methyl]phenyl]-2E-2-propenamide. As controls for pretreatment, new dilutions are added to untreated cells singly or in combination 48 hours after seeding.

The plates are incubated at 37° C. for 72 hours from seeding. The MTS solution is added (as for the T₀ plate) and read four hours later. In order to analyze the data, the average value of media alone (background) is subtracted from each experimental well and the triplicate values are averaged for each compound dilution. The following formulas are used to calculate percent growth.

The “% Growth” is plotted against compound concentration and used to calculate IC₅₀s employing the user-defined spline function in Microsoft Excel. This function uses linear regression between data points to predict the concentration of compounds at 50% inhibition. IC₅₀s are used to determine the dose range for each compound and the resultant combinations.

Using the CalcuSyn program, the Combination Index (CI) is determined by the isobologram equation CI=(D)₁/(Dₓ)₁+(D)₂/(Dₓ)₂. Drug 1 (D), and drug 2 (D)₂ in combination inhibit X % and (Dₓ)₁ and (Dₓ)₂ are the doses of drug 1 and drug 2 alone that also inhibits X %. For each compound the % growth values at each dose as determined in the MTS assay is used. CI values that are less than 1, equal to 1 or are greater than 1 indicate synergism, additive effect, or antagonism, respectively. CIs are compared at the following percent inhibitory concentrations: IC₂₅, IC₅₀, IC₇₅, and IC₉₀.

Results:

### Combination of N-hydroxy-3-[4-[[(2-hydroxyethyl)[2-(1H-indol-3-yl)ethyl]-amino]methyl]phenyl]-2E-2-propenamide with adriamycin

The antiproliferative effects of adding N-hydroxy-3-[4-[[(2-hydroxyethyl)[2-(1H-indol-3-yl)ethyl]-amino]methyl]phenyl]-2E-2-propenamide simultaneously, 24 hr after, or 24 hr before adding the chemotherapeutic agent adriamycin to the MDA-MB-435P cell line are examined. The combined effects are assessed using constant ratios of compound concentrations that are 8-fold, 4-fold, 2-fold, 1-fold, 0.5-fold, 0.25-fold and 0.125-fold of their respective IC₅₀s. To examine whether the combinations are additive, synergistic or antagonistic, isobolograms are plotted and combination indices calculated using the commercial software program CalcuSyn. In isobolograms, the X intercepts indicate the concentrations of one drug which results in a given percentage of growth inhibition and the Y intercepts indicate the concentrations at which the other drug inhibited the growth of the cells. The data point that falls between the axes indicates the concentration of the drug combination that inhibits cell growth. The farther above or below this data point deviates from the straight line joining the intercepts, the more antagonistic or synergistic the effect, respectively. Combination data points that fall on or close to the line joining the intercepts indicate additive effects.

Simultaneous incubation of MDA-MB-435P cells with adriamycin and N-hydroxy-3-[4-[[(2-hydroxyethyl)[2-(1H-indol-3-yl)ethyl]-amino]methyl]phenyl]-2E-2-propenamide or treatment with N-hydroxy-3-[4-[[(2-hydroxyethyl)[2-(1H-indol-3-yl)ethyl]-amino]methyl]phenyl]-2E-2-propenamide 24 hr prior to adding adriamycin produces isobologram combination data points close to the line joining the X and Y intercepts. The calculated combination indices are close to 1, indicating additive effects.

However, treatment of MDA-MB-435P cells with adriamycin 24 hr prior to N-hydroxy-3-[4-[[(2-hydroxyethyl)[2-(1H-indol-3-yl)ethyl]-amino]methyl]phenyl]-2E-2-propenamide results in combination data points far below the line joining the intercepts, indicating strong synergy between the two drugs. The above isobologram results are confirmed by calculation of combination indices.

Simultaneous addition of N-hydroxy-3-[4-[[(2-hydroxyethyl)[2-(1H-indol-3-yl)ethyl]-amino]methyl]phenyl]-2E-2-propenamide and adriamycin or pretreatment with the HDAI followed by adriamycin, results in combination indices close to 1, suggesting additive effects. However, pretreatment with adriamycin followed by N-hydroxy-3-[4-[[(2-hydroxyethyl)[2-(1H-indol-3-yl)ethyl]-amino]methyl]phenyl]-2E-2-propenamide results in a combination index of <0.1, indicating strong synergy.

### Combination of N-hydroxy-3-[4-[[(2-hydroxyethyl)[2-(1H-indol-3-yl)ethyl]-amino]methyl]phenyl]-2E-2-propenamide with camptothecin or 5-FU

The HCT116 colorectal carcinoma cell line is treated with N-hydroxy-3-[4-[[(2-hydroxyethyl)[2-(1H-indol-3-yl)ethyl]-amino]methyl]phenyl]-2E-2-propenamide in combination with 5-fluorouracil (5-FU) or camptothecin. As described above HCT116 cells are incubated with constant ratios of the IC₅₀ values of the individual compounds and the combined effect determined by plotting isobolograms and calculating combination indices. Combinations of N-hydroxy-3-[4-[[(2-hydroxyethyl)[2-(1H-indol-3-yl)ethyl]-amino]methyl]phenyl]-2E-2-propenamide with 5-FU produces an isobologram indicating additivity. Calculated combination indices also indicate additive effects with the N-hydroxy-3-[4-[[(2-hydroxyethyl)[2-(1H-indol-3-yl)ethyl]-amino]methyl]phenyl]-2E-2-propenamide in combination with 5-FU, or additive or weak antagonism with camptothecin on HCT116 cells.

### Combination of N-hydroxy-3-[4-[[(2-hydroxyethyl)[2-(1H-indol-3-yl)ethyl]-amino]methyl]phenyl]-2E-2-propenamide with 5-azacytidine

5-azacytidine is a non-reversible DNA methyltransferase inhibitor. Recent published results indicate a cross-talk between histone deacetylases and DNA methyl transferases. Therefore the combined effect of N-hydroxy-3-[4-[[(2-hydroxyethyl)[2-(1H-indol-3-yl)ethyl]-amino]methyl]phenyl]-2E-2-propenamide and 5-azacytidine is assessed. A concentration-dependent effect is seen at all drug addition schedules. A trend where antagonistic effects at low inhibitory concentrations (IC₂₅) and additive effects at high inhibitory concentrations (IC₅₀) is observed.

Discussion:

This example reports the effect of combining N-hydroxy-3-[4-[[(2-hydroxyethyl)[2-(1H-indol-3-yl)ethyl]-amino]methyl]phenyl]-2E-2-propenamide with standard chemotherapeutics and other compounds that affect chromatin structure on monolayer cell proliferation. There is very strong synergy between adriamycin when MDA-MB-435P cells are incubated with this cytotoxic drug 24 hr prior to addition of N-hydroxy-3-[4[[(2-hydroxyethyl)[2-(1H-indol-3-yl)ethyl]-amino]methyl]phenyl]-2E-2-propenamide.

Combination of N-hydroxy-3-[4-[[(2-hydroxyethyl)[2-(1H-indol-3-yl)ethyl]-amino]methyl]phenyl]-2E-2-propenamide with camptothecin or 5-FU on HCT116 colon carcinoma cells produces additive or weak antagonistic effects.

5Azacytidine/N-hydroxy-3-[4-[[(2-hydroxyethyl)[2-(1H-indol-3-yl)ethyl]-amino]methyl]phenyl]-2E-2-propenamide combinations exhibit concentration-dependent effects.

The basis for the synergism observed when MDA-MB-435P cells are pretreated with adriamycin followed by N-hydroxy-3-[4-[[(2-hydroxyethyl)[2-(1H-indol-3-yl)ethyl]-amino]methyl]phenyl]-2E-2-propenamide is currently not known. However, a situation where perturbation of chromatin structure by N-hydroxy-3-[4-[[(2-hydroxyethyl)[2-(1H-indol-3-yl)ethyl]-amino]methyl]phenyl]-2E-2-propenamide is exacerbated by DNA intercalation and induction of DNA damage by adriamycin can be envisaged.

The above combination studies identify effective therapeutic combinations and also help to avoid possible adverse drug interactions in clinical trials of N-hydroxy-3-[4-[[(2-hydroxyethyl)[2-(1H-indol-3-yl)ethyl]-amino]methyl]phenyl]-2E-2-propenamide.

1-37. (canceled)

38. A combination which comprises (a) a chemotherapeutic agent which is selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, discodermolides and epothilones such as epothilone B or D, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; pharmaceutically acceptable salts thereof; and pharmaceutically acceptable prodrugs thereof; and (b) a histone deacetylase inhibitor according to formula (I) 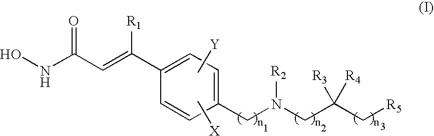
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
in which (a) and (b) are present in each case in free form or in the form of a pharmaceutically acceptable salt or a pharmaceutically acceptable prodrug thereof, for simultaneous, concurrent, separate or sequential use.

39. The combination of claim 38 wherein the histone deacetylase inhibitor is a compound of formula (I) wherein each of R₁, X, Y, R₃, and R₄ is H, or a pharmaceutically acceptable salt thereof or a pharmaceutically acceptable prodrug thereof.

40. The combination of claim 39 wherein the histone deacetylase inhibitor is a compound of formula (I) wherein one of n₂ and n₃ is zero and the other is 1, or a pharmaceutically acceptable salt thereof or a pharmaceutically acceptable prodrug thereof.

41. The combination of claim 40 wherein the histone deacetylase inhibitor is a compound of formula (I) wherein R₂ is H or —CH₂—CH₂—OH, or a pharmaceutically acceptable salt thereof or a pharmaceutically acceptable prodrug thereof.

42. The combination of claim 38 wherein the histone deacetylase inhibitor is a compound of the formula (Ia) 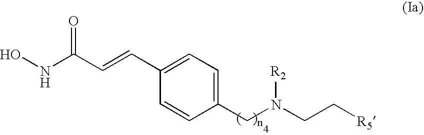
wherein
n₄ is 0-3,
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is heteroaryl, heteroarylalkyl, an aromatic polycycle, a non-aromatic polycycle, a mixed aryl and non-aryl polycycle, polyheteroaryl, or a mixed aryl and non-aryl polyheterocycle
or a pharmaceutically acceptable salt thereof or a pharmaceutically acceptable prodrug thereof.

43. The combination of claim 38 wherein the histone deacetylase inhibitor is a compound of the formula (Ib) 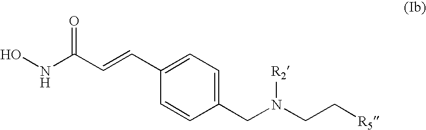
wherein
R₂′ is selected from H, C₁-C₆ alkyl, C₄-C₆ cycloalkyl, cycloalkylalkyl, and (CH₂)₂₋₄OR₂₁ where
R₂₁ is H, methyl, ethyl, propyl, or isopropyl, and
R₅″ is unsubstituted or substituted 1H-indol-3-yl, benzofuran-3-yl or quinolin-3-yl
or a pharmaceutically acceptable salt thereof or a pharmaceutically acceptable prodrug thereof.

44. The combination of claim 38 wherein the histone deacetylase inhibitor is a compound of the formula (Ie) 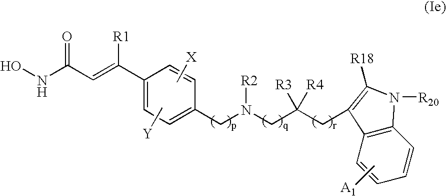
wherein
R18 is H, halo, C₁-C₆alkyl, C₃-C₇cycloalkyl, aryl, or heteroaryl;
R₂₀ is H, C₁-C₆alkyl, C₃-C₉cycloalkyl-C₁-C₆alkyl, aryl, heteroaryl, arylalkyl, heteroarylalkyl, acyl or sulfonyl;
A₁ is 1, 2 or 3 substituents which are independently H, C₁-C-₆alkyl, —OR₁₉, halo, alkylamino, aminoalkyl, halo, or heteroarylalkyl;
R₁₉ is selected from H, C₁-C₆alkyl, C₄-C₉cycloalkyl, C₄-C₉heterocycloalkyl, aryl, heteroaryl, arylalkyl, heteroarylalkyl and —(CH₂CH═CH(CH₃)(CH₂))₁₋₃H;
p is 0-3, and
q is 1-5 and r is 0 or
q is 0 and r is 1-5,
or a pharmaceutically acceptable salt thereof or a pharmaceutically acceptable prodrug thereof.

45. The combination of claim 44 wherein R₁₈ is H, fluoro, chloro, bromo, a C₁-C₄ alkyl group, a C₃-C₇ cycloalkyl group, phenyl or a heteroaryl ring, or a pharmaceutically acceptable salt thereof or a pharmaceutically acceptable prodrug thereof.

46. The combination of claim 45 wherein R₂ is H, or —(CH₂)ₛCH₂OH and wherein s is 1-3, or a pharmaceutically acceptable salt thereof or a pharmaceutically acceptable prodrug thereof.

47. The combination of claim 46 wherein R₁ is H and X and Y are each H, and wherein q is 1-3 and r is 0 or wherein q is 0 and r is 1-3, or a pharmaceutically acceptable salt thereof or a pharmaceutically acceptable prodrug thereof.

48. The combination of claim 38 wherein the histone deacetylase inhibitor is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)ethyl]-amino]methyl]phenyl]-2E-2-propenamide, or a pharmaceutically acceptable salt thereof or a pharmaceutically acceptable prodrug thereof.

49. The combination of claim 48 wherein the histone deacetylase inhibitor is selected from the group consisting of N-hydroxy-3-[4-[[(2-hydroxyethyl)[2-(1H-indol-3-yl)ethyl]-amino]methyl]phenyl]-2E-2-propenamide, or a pharmaceutically acceptable salt thereof or a pharmaceutically acceptable prodrug thereof.

50. A pharmaceutical composition which comprises (a) a chemotherapeutic agent, or a pharmaceutically acceptable salt thereof or a pharmaceutically acceptable prodrug thereof, and (b) a histone deacetylase inhibitor, or a pharmaceutically acceptable salt thereof or a pharmaceutically acceptable prodrug thereof, together with at least one pharmaceutically acceptable carrier.

51. The pharmaceutical composition of claim 50 wherein the chemotherapeutic agent is selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, discodermolides and epothilones such as epothilone B or D, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; or a pharmaceutically acceptable salt thereof or a pharmaceutically acceptable prodrug thereof.

52. The pharmaceutical composition of claim 50 wherein the histone deacetylase inhibitor is selected from a histone deacetylase inhibitor according to formula (I) 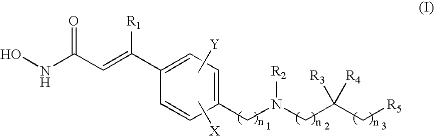
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂),OC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
in which (a) and (b) are present in each case in free form or in the form of a pharmaceutically acceptable salt or a pharmaceutically acceptable prodrug thereof, for simultaneous, concurrent, separate or sequential use.

53. A commercial package or product comprising a chemotherapeutic agent, or a pharmaceutically acceptable salt thereof or a pharmaceutically acceptable prodrug thereof, together with instructions for use in combination with a histone deacetylase inhibitor, or a pharmaceutically acceptable salt thereof or a pharmaceutically acceptable prodrug thereof, for the treatment of a disease in a mammal, or
a commercial package or product comprising a histone deacetylase inhibitor, or a pharmaceutically acceptable salt thereof or a pharmaceutically acceptable prodrug thereof, together with instructions for use in combination with a chemotherapeutic agent, or a pharmaceutically acceptable salt thereof or a pharmaceutically acceptable prodrug thereof, for the treatment of a disease in a mammal.

54. The commercial package or product of claim 53 wherein the chemotherapeutic agent is selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, discodermolides and epothilones such as epothilone B or D, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; or a pharmaceutically acceptable salt thereof or a pharmaceutically acceptable prodrug thereof.

55. The commercial package or product of claim 53 wherein the histone deacetylase inhibitor is selected from a histone deacetylase inhibitor according to formula (I) 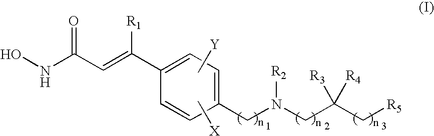
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
in which (a) and (b) are present in each case in free form or in the form of a pharmaceutically acceptable salt or a pharmaceutically acceptable prodrug thereof, for simultaneous, concurrent, separate or sequential use.

56. A commercial package or product comprising a combination according to claim 38 together with instructions for simultaneous, concurrent, separate or sequential use thereof in the treatment of a disease in a mammal.

57. The commercial package or product according to claim 56 wherein the disease is a proliferative disease.

58. The commercial package or product of claim 56 wherein the proliferative disease is selected from the group consisting of breast cancer, lung cancer, ovarian cancer, lymphoma, head and neck cancer and cancer of the esophagus, stomach, bladder, prostrate, uterus and cervix.

59. A combined preparation which comprises (a) one or more unit dosage forms of a chemotherapeutic agent, or a pharmaceutically acceptable salt thereof or a pharmaceutically acceptable prodrug thereof, and (b) one or more unit dosage forms of a histone deacetylase inhibitor, or a pharmaceutically acceptable salt thereof or a pharmaceutically acceptable prodrug thereof.

60. The combined preparation of claim 59 wherein the chemotherapeutic agent is selected from the group consisting of DNA topoisomerase I inhibitors; DNA topoisomerase II inhibitors; microtubule active agents; and antimetabolites including agents which are inhibitors of thymidine production, inhibitors of vascular endothethial growth factor, DNA demethylating agents, or protein-tyrosine kinase inhibitors, such as e.g., Adriamycin, discodermolides and epothilones such as epothilone B or D, 5-Fluorouracil, camptothecin or derivatives thereof such as gimatecan, Imatinib (Gleevec), 1-[4-chloroanilino]-4-[pyridylmethyl]-phthalazine succinate (PTK787), 5-Aza dC (Decitabine) and 5-Azacytidine; or a pharmaceutically acceptable salt thereof or a pharmaceutically acceptable prodrug thereof.

61. The combined preparation of claim 59 wherein the histone deacetylase inhibitor is selected from a histone deacetylase inhibitor according to formula (I) 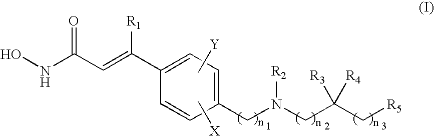
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
in which (a) and (b) are present in each case in free form or in the form of a pharmaceutically acceptable salt or a pharmaceutically acceptable prodrug thereof, for simultaneous, concurrent, separate or sequential use.

62. A method for the prevention or treatment of proliferative diseases in a mammal, which comprises treating the mammal with pharmaceutically effective amounts of a combination according to claim 38.

63. The method of claim 62 wherein the proliferative disease is selected from the group consisting of breast cancer, lung cancer, ovarian cancer, lymphoma, head and neck cancer and cancer of the esophagus, stomach, bladder, prostrate, uterus and cervix.

64. The method of claim 62 wherein the proliferative diseases is selected from pre-malignant lesions, solid malignancies and undifferentiated malignancies.

65. The method of claim 62 wherein the histone deacetylase inhibitor is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)ethyl]-amino]methyl]phenyl]-2E-2-propenamide, or a pharmaceutically acceptable salt thereof or a pharmaceutically acceptable prodrug thereof.
